(19) United States
(12) Patent Application Publication
(10) Pub. No.: US 2003/0039955 A1
(43) Pub. Date: Feb. 27, 2003

(54) Compositions and methods for production of RNA viruses and RNA virus-based vector particles

(76) Inventors: Yu Feng, San Diego, CA (US); Hengli Tang, Carlsbad, CA (US)

Correspondence Address: GREGORY P. EINHORN, Fish & Richardson P.C., Suite 500, 4350 La Jolla Village Drive, San Diego, CA, 92122, US

(21) Appl. No.: 09/853,745

(22) Filed: May 10, 2001

Related U.S. Application Data
(60) Provisional application No. 60/206,997, filed on May 24, 2000.

(51) Int. Cl. C12N 7/00; C07H 21/04; C12N 15/86; C12N 15/867; C12N 15/863; C12Q 1/70; C12N 7/01; C12N 15/00; C12N 15/09; C12N 15/63; C12N 15/70; C12N 15/74

(52) U.S. Cl. 435/5; 435/235.1; 435/320.1; 435/456; 536/23.72

(57) ABSTRACT

The invention provides methods to produce RNA viral sequences, recombinant RNA viruses, mutants of RNA viruses and RNA virus-derived vectors in cell culture and in vitro using non-viable, replication defective, helper vaccinia recombinants. These methods allow generation of RNA virus sequences and viral particles in cell culture and in vitro independent of their natural replication pathways, bypassing the limitation of any cellular barriers. The invention also provides novel RNA viral sequences and viral particles using these methods.

### RELATED APPLICATIONS

This application claims the benefit of priority under 35 U.S.C. §119(e) of U.S. Provisional Application No. 60/206,997, filed May 24, 2000. The aforementioned application is explicitly incorporated herein by reference in its entirety and for all purposes.

### TECHNICAL FIELD

This invention generally pertains to the fields of virology, medicine and gene therapy. The present invention pertains to the methods for production of recombinant hepatitis C virus (HCV), rhinovirus, influenza virus and lentivirus-derived vector particles using non-infectious helper vaccinia virus. The compositions and methods of the invention are used to make replication defective gene therapy vector preparations that are substantially free of replication competent helper viruses.

### BACKGROUND

Viruses having genomes consisting of RNA, such as hepatitis C virus (HCV), retrovirus, rhinovirus, influenza virus and lentivirus, are potential sources of vaccine and gene therapy vectors. Attenuated viruses do not cause diseases, but selection and production of attenuated viruses are often difficult because of lack of culture systems to grow the impaired mutants. RNA virus-derived vectors, as other gene therapy vectors, contain expression cassettes for foreign genes. The vectors can be packaged into viral particles and delivered into target cells upon infection. The use of RNA viruses as gene therapy vectors has been impeded by their poor packaging efficiencies in cell culture systems.

### SUMMARY

The invention provides novel methods for producing RNA viral genomic sequences and recombinant RNA viruses and virus-derived vectors in cell culture or in vitro using non-viable, replication defective, helper poxvirus recombinants. These methods generate RNA viral genomes and viral particles in cell culture and in vitro independent of their natural replication pathways, bypassing the limitation of any cellular barriers. The invention also provides novel viral sequences using these methods.

The invention provides a method for producing an encapsidated RNA virus, comprising the following steps: (a) providing polypeptide coding sequences, wherein the polypeptides are capable of forming a capsid and packaging an RNA virus genomic sequence in a eukaryotic cell; (b) providing a construct comprising RNA virus genomic sequences operably linked to a bacteriophage promoter and a bacteriophage transcription termination sequence, wherein the bacteriophage promoter and the bacteriophage transcription termination sequence are operably compatible; (c) providing a coding sequence for a bacteriophage polymerase operably compatible with the bacteriophage promoter of step (b), wherein the coding sequence is operably linked to a poxvirus promoter; and, (d) expressing the polypeptides of step (a), the RNA virus genomic sequences of step (b) and the coding sequence for a bacteriophage polymerase of step (c) together in a eukaryotic cell cytoplasm under conditions allowing for the expression of the sequences and assembly of a capsid comprising the RNA virus genomic sequences, thereby making an encapsidated RNA virus.

In alternative aspects of the method, the genes encoding the capsid-forming polypeptides are cloned into a plasmid or a viral vector, particularly if the construct of step (b) (i.e., a construct comprising an RNA virus genomic sequences operably linked to a bacteriophage promoter and transcription termination sequence) has no functional internal ribosomal entry site (IRES). The coding sequences of step (a) can be operably linked to a promoter that is active in a eukaryotic, e.g., an animal, such as a mammalian, cell cytoplasm.

In one aspect, the coding sequence for the bacteriophage polymerase is cloned into a replication defective poxvirus; this coding sequence can be operably compatible with the bacteriophage promoter of step (b).

In one aspect of the method, the construct comprising RNA virus genomic sequences can comprise a plasmid or a viral vector.

In one aspect of the method, the bacteriophage is selected from the group consisting of a T3 bacteriophage, a T7 bacteriophage and an SP6 bacteriophage. The T3 bacteriophage polymerase can be expressed with a T3 bacteriophage promoter, a T7 bacteriophage polymerase can be expressed with a T7 bacteriophage promoter and an SP6 bacteriophage polymerase can be expressed with an SP6 bacteriophage promoter. The construct can comprise a T3 bacteriophage transcription termination sequence and a T3 bacteriophage promoter, a T7 bacteriophage transcription termination sequence and a T7 bacteriophage promoter, or, an SP6 bacteriophage transcription termination sequence and a SP6 bacteriophage promoter.

In one aspect of the method, the promoter active in a eukaryotic cell cytoplasm can be a promoter derived from a virus of the family Poxviridae. The virus of the family Poxviridae can be a virus of the genus Orthopoxvirus. The virus of the genus Orthopoxvirus can be a vaccinia virus. The vaccinia virus promoter can be a late vaccinia virus promoter, an intermediate vaccinia virus promoter or an early vaccinia virus promoter. The poxvirus can be a virus of the Orthopoxvirus genus, such as a vaccinia virus. Alternatively, the poxvirus can be a virus of a genus selected from the group consisting of a Parapoxvirus genus, Avipoxvirus genus, a Capripoxvirus genus, Yatapoxvirus genus, a Leporipoxvirus genus, a Suipoxvirus genus and a Molluscipoxvirus genus.

In alternative aspects of the method, the eukaryotic cell cytoplasm comprises a eukaryotic cell, or, the eukaryotic cell cytoplasm comprises an in vitro preparation.

In one aspect of the method, the RNA virus is a hepatitis virus, such as a hepatitis A virus, any hepatitis B virus with an RNA genome, an immature hepatitis B virus that comprises a pre-genomic RNA in its core, or a hepatitis C virus. Alternatively, the RNA virus can be a lentivirus, a rhinovirus, an influenza virus, a human immunodeficiency virus (HIV), such as HIV-1 (in one embodiment, the human immunodeficiency virus lacks a Rev-responsive element or an envelope sequence), an arenavirus, a LCMV, a parainfluenza virus, a reovirus, a rotavirus, an astrovirus, a filovirus, or a coronavirus (see discussion below, as the invention includes all RNA viruses).

In alternative aspects of the method, the replication defective, encapsidated RNA virus is infectious, or, is non-infectious.

In alternative aspects of the invention, the method produces a preparation that is substantially free of replication competent poxvirus, for example, the method produces a preparation that is 99% free of replication competent poxvirus, 99.5% free of replication competent poxvirus or 100% free of replication competent poxvirus (see definition of “substantially free,” below).

In one aspect, the replication defective poxvirus lacks the ability to make a polypeptide necessary for viral replication. The polypeptide necessary for viral replication can be a viral capsid polypeptide. The replication defective poxvirus can be defective because of a transcriptional activation or a transcriptional regulation defect.

In one aspect, one, several or all of the polypeptide coding sequences of step (a) are incorporated into the RNA virus genomic sequence of step (b) and the construct further comprises an internal ribosomal entry site (IRES). IRES can be derived from any source, as discussed in detail, below.

The invention provides a system for producing an encapsidated RNA virus, comprising the following components: (a) polypeptide coding sequences, wherein the polypeptides are capable of packaging an RNA virus genomic sequences and each coding sequence is cloned into a construct such that it is operably linked to a promoter; (b) a construct comprising RNA virus genomic sequence operably linked to a bacteriophage promoter and a bacteriophage transcription termination sequence, wherein the RNA virus genomic sequence can be packaged into a capsid by the polypeptides of step (a); (c) a coding sequence for a bacteriophage polymerase operably compatible with the bacteriophage promoter of step (b), wherein the coding sequence is cloned into a replication defective poxvirus such that the coding sequence is operably linked to a poxvirus promoter; and, wherein expressing the polypeptides of step (a), the RNA virus genomic sequence of step (b) and the coding sequence for a bacteriophage polymerase of step (c) together in a eukaryotic cell cytoplasm under conditions allowing for the expression of the coding sequences and assembly of a capsid comprising the RNA viral genomic sequence produces an encapsidated RNA virus.

In one aspect of the system, one, several or all of the polypeptide coding sequences of step (a) are incorporated into the RNA virus genomic sequence of step (b) and the construct further comprises an internal ribosomal entry site (IRES).

The invention provides a recombinant viral genomic sequence comprising an RNA genomic sequence and a 2′,3′ cyclic phosphate at its 3′ end. The invention provides a recombinant viral particle comprising an RNA genomic sequence and a 2′,3′ cyclic phosphate at its 3′ end. The RNA genomic sequence can be derived from any RNA virus, as discussed in detail, below.

The invention provides a recombinant viral genomic sequence comprising an RNA genomic sequence and a transcriptional terminator sequence for a bacteriophage RNA polymerase followed by a poly A sequence at its 3′ end. The invention provides a recombinant viral particle comprising an RNA genomic sequence and a transcriptional terminator sequence for a bacteriophage RNA polymerase followed by a poly A sequence at its 3′ end. The RNA genomic sequence can be derived from any RNA virus, as discussed in detail, below. In one aspect, the genomic sequence is encapsidated.

The invention provides a recombinant lentivirus genomic sequence lacking a Rev-response element (RRE) or an envelope sequence and comprising a terminator sequence for a bacteriophage RNA polymerase. The invention provides a recombinant lentivirus particle comprising an RNA genomic sequence lacking a Rev-response element (RRE) or an envelope sequence and comprising a terminator sequence for a bacteriophage RNA polymerase.

All publications, patents and patent applications cited herein are hereby expressly incorporated by reference for all purposes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates plasmid pT7HCV, which contains a DNA copy of the HCV genome, as described in detail in Example 1, below.

FIG. 2 illustrates plasmid pVHCV, which contains a HCV polyprotein-coding region, as described in detail in Example 1, below.

FIG. 3 illustrates plasmid pVAC, as described in detail in Example 1, below.

FIG. 4 illustrates plasmid pT7HCV-RIB, containing a DNA copy of the HCV genomic RNA, a hairpin ribozyme (Rz) flanked by a bacteriophage T7 promoter (PT7) and a bacteriophage T7 terminator (TT7), as described in detail in Example 1, below.

FIG. 5 illustrates plasmid pRHIN; in this plasmid, the OUF of rhinovirus polyprotein is flanked by a vaccinia late promoter and a vaccinia terminator, as described in detail in Example 2, below. The thin lines represent the pUC19 backbone.

FIG. 6 illustrates plasmid pT7RHIN; in this plasmid, a T7 promoter is followed by a DNA copy of the rhinovirus genomic RNA, which include the 5′ UTR, the polyprotein-coding region and the 3′ UTR followed by poly(A) and the cDNA of a hairpin-ribozyme (Rz) followed by a T7 terminator, as described in detail in Example 2, below. The thin lines represent the pUC19 backbone.

FIG. 7 illustrates plasmid pINF1-8; in this plasmid, the ORFs of influenza A NS and PB2 are linked to two separate vaccinia late promoters, as described in detail in Example 3, below. The arrow indicates the direction of transcription. The thin line indicates the pUC19 backbone.

FIG. 8 illustrates plasmid pT7INF1; in this plasmid, the cDNA of the segment 1 RNA of influenza A is linked to a hairpin-ribozyme-coding sequence, as described in detail in Example3, below. The entire region is flanked by a T7 promoter and a T7 terminator.

FIG. 9 illustrates plasmid pGAG-POL; in this plasmid, the HIV-1 HXB2 gag/pol polyprotein-coding region is flanked by a vaccinia early/later promoter (PvacE/L) and a vaccinia terminator (Tvac), as described in detail in Example 4, below. The thin lines represent the pUC19 backbone.

FIG. 10 illustrates plasmid pVSVG; in this plasmid, the vesicular stomatitis virus G (VSV-G) protein-coding region is flanked by a bacteriophage T7 promoter (PT7) and a bacteriophage T7 terminator (TT7), as described in detail in Example 4, below. The thin lines represent the pT7 backbone.

FIG. 11 illustrates plasmid pT7EGFP; in this plasmid, a bacteriophage T7 promoter (PT7) is followed by a triple nucleotide G followed by the HIV-1 HXB2 5′ LTR followed by the HIV-1 HXB2 packaging signal followed by the cytomegalovirus (CMV) promoter followed by the enhanced green fluorescent protein-coding region followed by the HIV-1 HXB2 polypurine tract (PPT) followed by the HIV-1 HXB2 3′ U3 followed by a triple nucleotide G followed by HIV-1 HXB2 3′ R followed by a bacteriophage T7 terminator (TT7), as described in detail in Example 4, below. The thin lines represent the pBR322 backbone.

Like reference symbols in the various drawings indicate like elements.

### DETAILED DESCRIPTION

It is an object of the present invention to provide methods of using non-viable, i.e., replication defective, recombinant poxvirus to produce high titer preparations of encapsidated RNA genomic sequences and RNA virus vectors, and RNA virus particles. The RNA viruses and genomic sequences can be any RNA virus, including, for example, hepatitis viruses (e.g., hepatitis C, HCV), rhinoviruses, influenza viruses and lentiviruses. The RNA virus vectors and encapsidated products produced using these methods are substantially, or completely, free of infectious poxvirus. The methods provided by this invention can also be used to produce any RNA virus.

In one aspect of the invention, methods for production of RNA viruses (e.g., HCV, rhinoviruses and influenza viruses) comprise the steps of: (a) co-transfecting cells with a plasmid containing a viral genomic RNA-coding region between a bacteriophage promoter and a bacteriophage transcriptional terminator and plasmids containing transcription units for viral proteins, (b) infecting said cells with a non-viable poxvirus recombinant that contains a bacteriophage RNA polymerase gene, (d) harvesting the RNA virus particles.

In one aspect of the invention, methods for producing lentiviral vector-particles comprise the steps of: (a) co-transfecting cells with a plasmid containing a lentivirus-derived vector-coding region between a bacteriophage promoter and a bacteriophage transcriptional terminator and plasmids containing transcription units for viral proteins, (b) infecting said cells with a nonviable poxvirus recombinant that contains a bacteriophage RNA polymerase gene, (d) harvesting the vector particles.

The invention also provides infectious poxvirus-free preparations of RNA viruses (e.g., HCV, rhinoviruses, influenza viruses) that contain virion RNA with a terminator sequence for bacteriophage RNA polymerase or with a 2′,3′-cyclic phosphate 3′ terminus.

The invention also provides infectious poxvirus-free preparations of lentiviral vector-particles that contain a vector without the Rev-response element or any other envelope sequence and with a terminator sequence for bacteriophage RNA polymerase.

### Production of HCV, Rhinoviruses Influenza Viruses and Lentiviral Vectors

The replication-defective helper poxvirus used for production of the RNA viruses of the invention (e.g., HCV, rhinoviruses, influenza viruses and lentivirus-derived vectors) can be a vaccinia recombinant virus. The replication-defective poxvirus has a bacteriophage RNA polymerase gene inserted in the thymidine kinase-coding region of its genome. The expression of the RNA polymerase is driven by a poxvirus, e.g., a vaccinia, promoter. An exemplary method to generate the vaccinia recombinant containing a bacteriophage RNA polymerase gene was described by Fuerst (1986) Proc. Natl. Acad. Sci. USA 83:8122-8126.

In one aspect, in addition to the RNA polymerase gene, the replication-defective helper poxvirus (e.g., the helper vaccinia recombinant) has a replication defect, e.g., a defect in an essential gene, e.g., a deletion in an essential gene; or, has an inducible essential gene, or, has an essential gene under the control of a promoter for RNA polymerase which is not from poxvirus. For example, the D13L-defective vaccinia recombinant vT7ΔD13L can be used to produce RNA virus, such as HCV, rhinoviruses, influenza viruses and lentivirus-derived vectors. The D13L gene product is required for assembly of the virions, i.e., it is an essential gene. Inhibition or repression of its expression has no effect on viral transcription and DNA replication (see, e.g., Zhang (1992) Virol. 187:643-653), but formation of vaccinia virion is prevented. Thus use of D13L-negative vaccinia recombinant to produce RNA virus particles (e.g., HCV, rhinoviruses, influenza viruses and the lentiviral vector) can result in preparations with little contamination of helper vaccinia virus.

In the exemplary methods described below, construction and propagation of D13L-negative vaccinia recombinant was carried out according to Falkner, et al., (1998) U.S. Pat. No. 5,770,212, with some modifications. In the D13L-negative vaccinia recombinant, the D13L ORF was replaced by a bacterial guanine phosphoribosyltransferase (gpt) gene and a lacZ gene through homologous recombination. The expression of gpt and lacZ gene is controlled by a vaccinia early/late promoter. The defective vaccinia virus was selected and propagated in HeLa cells transiently transfected with a plasmid that encodes a D13L gene under the control of a vaccinia late promoter.

In addition to the defective vaccinia recombinant, conditional lethal, inducer-dependent vaccinia recombinants, or RNA polymerase (e.g., bacteriophage RNA polymerase) vaccinia recombinants, can also be used in the methods of the invention for the production of RNA viruses. One of such recombinants contains the IPTG-inducible D13L gene (see, e.g., Zhang (1992) Virol. 187:643-653). In the absence of IPTG, reproduction of the vaccinia recombinant is suppressed. Alternatively, the vaccinia promoter of the D13L gene can be replaced by a bacteriophage promoter. If the promoter for the D13L gene is a bacteriophage promoter, without the bacteriophage RNA polymerase, the D13L gene product cannot be produced.

In one aspect of the invention, to generate HCV from the cloned cDNA, two plasmids are used. One contains a DNA copy of a full length HCV genomic RNA that is cloned between a bacteriophage promoter (e.g., T7, SP6 or T3 promoter) and a bacteriophage transcription terminator. Transcription of such a transcription unit by a bacteriophage RNA polymerase that recognizes the promoter and terminator will generate RNA molecules with a defined size. The other plasmid contains the coding region of the viral polyprotein directly linked to an upstream vaccinia late promoter. These plasmids are used to co-transfect suitable host cells which are easily transfected and susceptible to vaccinia viruses. The transfected host cells are then infected with a helper vaccinia recombinant that contains a bacteriophage RNA polymerase gene under the control of a vaccinia promoter, e.g., the vaccinia late or early/late promoter.

In one aspect, the helper vaccinia recombinant also contains a defect in a gene necessary for replication or encapsidation, i.e., an essential gene, or, has an inducible essential gene. For example, after 72 to 96 hours incubation at 30° C., the cell culture medium is collected and filtered through a 0.2 μm filter to remove residual vaccinia viral particles. The filtrate contains HCV virions. The HCV particles produced using the method provided by this invention resemble the natural virions but their virion RNA molecules are different from the natural ones. They contain a terminator sequence for bacteriophage RNA polymerase at the 3′ end, and approximately one half of the RNA molecules have a poly(A) tract following the terminator sequence. At the 5′ end, the virion RNA may have up to three extra nucleotides, and 5 to 10% of the RNA has a cap. This HCV preparation is able to infect MT-2 and Huh7 cells, generating the negative strand RNA.

To obtain RNA genomic sequence (e.g., HCV particles) in which the virion RNA does not contain a bacteriophage transcription termination sequence (e.g., a T7 terminator sequence), a plasmid containing a hairpin-ribozyme cassette (see, e.g., Altschuler (1992) Gene 122:85-90) is used for in vivo synthesis of virion (e.g., HCV) RNA. In the plasmid, the 3′ end of the cDNA which encodes virion RNA is ligated to a hairpin-ribozyme cDNA (see FIG. 4). The DNA that has the virion RNA-ribozyme-coding sequence is then placed between a bacteriophage promoter and a bacteriophage terminator. Following transcription, the resulting transcripts will be auto-cleaved by the cis-cleavage reaction carried out by the hair-pin ribozyme to generate virion RNA with no bacteriophage terminator sequence at the 3′ end. The resulting virion RNA is structurally distinguished by its 3′ terminus of 2′,3′ cyclic phosphate. When this construct was used to express HCV virion RNA, an increase in the titer of the resulting viral particles was observed.

In one exemplary method for generating rhinovirus, two plasmids are used. One contains a DNA segment that consists of the cDNA of the virion RNA followed by a 70 nucleotides of poly(A) tract followed by the cDNA of a hairpin-ribozyme. The DNA segment is flanked by a bacteriophage promoter and a bacteriophage terminator. The other plasmid contains the RNA virus (e.g., rhinovirus) polyprotein-coding region downstream of a vaccinia late promoter. These two plasmids are used to co-transfect cells that are susceptible to both vaccinia virus and other RNA viruses, such as rhinovirus. Next, the transfected cells are infected with the helper vaccinia recombinants that contain a bacteriophage RNA polymerase gene under the control of a vaccinia late or early/late promoter. After incubation at 30° C. for 72-96 hours, the cell culture supernatant is collected and filtered through a 0.2 μm filter. The filtrate contains infectious RNA virus. The virions generated contained an RNA molecule with a 2′,3′ cyclic phosphate at the 3′ terminus.

In one exemplary method to generate influenza virus, two types of plasmids are needed. One consists of the cDNA of the virion RNA followed by the cDNA of a hairpin-ribozyme (see, e.g., Chowrira (1994) J. Biol. Chem. 269: 25856-25864). The cDNA is placed between a bacteriophage promoter and a bacteriophage terminator. Influenza A and B have eight segments of single strand and negative sense RNA, and influenza C has seven. In order to express a whole set of the segments, eight plasmids are constructed such that each plasmid encodes one RNA segment. The other type of plasmids contains the coding regions for the viral proteins (PB1, PB2, PA, HA, NP, NA, M, and NS) downstream of a vaccinia late promoter. Each plasmid encodes two viral proteins. Cells that are susceptible to both vaccinia virus and influenza are co-transfected with the twelve plasmids (eight for the genomic RNA segments and four for the viral proteins) followed by infection with the helper vaccinia recombinants that contains a bacteriophage RNA polymerase gene. After incubation at 30° C. for 72-96 hours, the culture supernatant is collect and filtered. The filtrate contains influenza virus. The virions generated contained a virion RNA with a 3′ terminus of 2′,3′ cyclic phosphate.

In one exemplary method to generate lentivirus-derived vector particles, three plasmids are needed. One contains cDNA encoding the vector RNA between a bacteriophage promoter and a corresponding transcriptional terminator. The DNA segment comprises coding regions of a 5′ long terminal repeat (LTR), a packaging signal, a desired protein ORF linked to a proper promoters (e.g., CMV, SV 40 promoters and other tissue specific promoters), a polypurine tract, and a 3′ LTR. Another plasmids contain cDNA encoding Gag-Pol protein for packaging. The third plasmid contains cDNA encoding a viral envelope protein for targeting and entry. The cDNAs are linked to a poxvirus, e.g., a vaccinia, promoter, e.g., a late promoter. Vaccinia susceptible cells are transfected with the plasmids and subsequently infected by the replication defective helper vaccinia recombinants that contain a bacteriophage RNA polymerase under the control of a vaccinia promoter (e.g., late or early/late promoter). After incubation at 30° C. for 72 to 96 hours, the vector particles are collected from the culture supernatant and filtered through a 0.2 μm filter. The vectors packaged in the particles contain a bacteriophage terminator sequence, and about a half of the vectors have a poly(A) tract following the bacteriophage terminator sequence. In one aspect, the vectors do not contain a cellular transport element (e.g., the Rev response element) that is required by other methods. This method can be used for a large-scale vector particle preparation. The titer of the preparation can reach 10⁸ cfu/ml.

RNA synthesis by a bacteriophage RNA polymerase is more efficient when the transcription starts with two or three Gs. Thus, in one aspect of the invention, the resulting transcripts are designed to comprise a double or triple G tag at the 5′ end of the transcripts. For some lentiviral vectors, a modification on the vector RNA may be necessary in order to allow reverse transcription to proceed. For example, the strong stop DNA reverse-transcribed from the vector RNA that is synthesized by T7 RNA polymerase will have two or three Gs at its 3′ end. In order to let the strong stop DNA form base-pairs with the 3′ LTR, certain number (one, two, or three) of Gs may be inserted between the U3 and R of the 3′ LTR (see, e.g., Coffin, Fields Virology, 3d., Philadelphia, N.Y.: Lippincott-Raven Publisher 1996, pp 1767-1847).

The incubation temperature following the infection of helper vaccinia virus is extremely important for the high yield production of the viruses and the vector particles. Although the optimal temperature for the replication of vaccinia virus is about 37° C., the optimal temperature for producing RNA virus and the vector particles is about 29° C.±2° C. For example, the HCV virions produced at 30° C. is 500 to 1,000 fold higher than is at 37° C. The HIV-derived vector particles produced at 30° C. is 10⁸ cfu/ml culture medium and about 1,000 fold higher than is produced at 37° C.

### Definitions

Unless defined otherwise, all technical and scientific terms used herein have the meaning commonly understood by a person skilled in the art to which this invention belongs. As used herein, the following terms have the meanings ascribed to them unless specified otherwise.

The term “RNA virus” refers to a virus whose genome comprises RNA. Specific examples of RNA viruses include all RNA genome-containing hepatitis viruses, including hepatitis A, immature hepatitis B, and hepatitis C (HCV), rhinoviruses, influenza viruses, arenaviruses, LCMV, parainfluenza viruses, reoviruses, rotaviruses, astroviruses, filoviruses, coronaviruses. The term “RNA virus” includes viruses of the family Retroviridae, such as viruses of the genus Lentivirus or Spumavirus, viruses of the family Totiviridae, viruses of the genus Tobravirus, deltaviruses, insect viruses such as Nyamanini virus. RNA viruses also include plant viruses, such as those found in the genus Furovirus, viruses of the genus Umbravirus, viruses of the family Sequiviridae, viruses of the genus Machlomovirus, viruses of the genus Iaedovirus and Viroids.

The term “poxvirus” refers to all viruses of the family Poxviridae, including viruses of the subfamily Chordopoxvirinae, such as viruses of the genus Orthopoxvirus (e.g., vaccinia virus), viruses of the genus Parapoxvirus, viruses of the genus Avipoxvirus, viruses of the genus Capripoxvirus, viruses of the genus Leporipoxvirus, viruses of the genus Molluscipoxvirus, viruses of the genus Suipoxvirus, viruses of the genus Yatapoxvirus; viruses of the subfamily Entomopoxvirinae, and other taxonomically unassigned viruses, such as the California harbor sealpox virus, cotia virus, Molluscum-likepox virus, mule deerpox virus, and the like.

The term “poxvirus promoter” includes any poxvirus promoter, many of which are known in the art. Poxviruses, e.g., vaccinia viruses, replicate in the cytoplasmic compartment of eukaryotic cells. Classes of poxvirus promoters include, for example, vaccinia early, intermediate and late promoters. See, e.g., Broyles (1997) J. Biol. Chem. 274:35662-35667; Zhu (1998) J. Virol. 72:3893-3899; Holzer (1999) Virology 253:107-114; Carroll (1997) Curr. Opin. Biotechnol. 8:573-577; Sutter (1995) FEBS Lett. 371:9-12. The term “promoter” is an array of nucleic acid control sequences which direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements that can be located as much as several thousand base pairs from the start site of transcription. A “constitutive” promoter is a promoter which is active under most environmental and developmental conditions. An “inducible” promoter is a promoter which is under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “defective poxvirus” refers to a poxvirus that contains a defect, a mutation or a recombinant manipulation in an essential gene (any gene required for replication or encapsidation) of its parental poxvirus. For example, the essential gene may engineered be under the control of an inducible promoter, or, under the control of a promoter that is only used by an RNA polymerase from a species other than a poxvirus. The term “non-viable poxvirus” refers to a poxvirus with a lethal or conditional lethal mutation or defect. The term “inducer-dependent, conditional lethal virus” refers to the mutants of viruses that contain inducible essential genes in the genome. The term “inducible essential genes” refers to the genes that are vital and expressed only in the presence of specific inducers. The term “replication deficient” or “replication defective” refers to a viral genome that does not comprise all the genetic information necessary for replication and formation of a genome-containing capsid under physiologic (e.g., in vivo) conditions.

The term “mutated RNA virus” refers to an RNA virus whose genomic RNA contains nucleotide sequences different from that of a corresponding wild type RNA virus. The term “recombinant RNA virus” refers to an RNA virus whose genome contains a sequence derived from other species or a sequence synthesized in vitro, or where genomic sequences have been manipulated, e.g., rearranged.

The term “RNA virus-derived vector” refers to RNA that contains an expression cassette(s) for foreign proteins and can be packaged into a viral particle. The term “vector RNA” refers to RNA that contains an expression cassette(s) for foreign proteins and can be packaged into a viral particle. The term “viral particle” refers to a virion in which all or some of a genomic nucleic acid of a virus is packaged. The term “vector particle” refers to a viral particle in which the nucleic acid encoding an expression cassette(s) is packaged.

The term “expression cassette” as used herein refers to a nucleotide sequence which is capable of affecting expression of a structural gene (i.e., a protein coding sequence) in a host compatible with such sequences. Expression cassettes include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid that can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include RNA replicons to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and nonexpression plasmids. Where a recombinant microorganism or cell culture is described as hosting an “expression vector” this includes both extrachromosomal circular and linear DNA and DNA that has been incorporated into the host chromosome(s). When a vector is maintained by a host cell, the vector may either be stably replicated by the cells during mitosis as an autonomous structure, or is incorporated within the host's genome.

The terms “bacteriophage promoter” and “bacteriophage transcription termination sequence” refers to any bacteriophage promoter or transcription termination sequence, respectively, many of which are well known in the art, including, e.g., promoters and termination sequences from T3 bacteriophage, T7 bacteriophage and SP6 bacteriophage. Methods for cloning and manipulating bacteriophage promoters and bacteriophage transcription termination sequences are well known in the art; see, e.g., Yoo (2000) Biomol. Eng. 16:191-197; Bermudez-Cruz (1999) Biochimie 81:757-764; Greenblatt (1998) Cold Spring Harb. Symp. Quant. Biol. 63:327-336; Cisneros (1996) Gene 181:127-133; and U.S. Pat. Nos: 6,143,518; 6,110,680; 6,096,523; 5,891,636; 5,792,625. The term “bacteriophage polymerase” refers to any bacteriophage polymerase, including those compatible with T3 bacteriophage, T7 bacteriophage and SP6 bacteriophage promoters. Methods for cloning and manipulating bacteriophage polymerases are well known in the art; see, e.g., Temiakov (2000) Proc. Natl. Acad. Sci. USA 97:14109-14114; Pavlov (2000) Nucleic Acids Res. 28:4657-4664; Jeng (1997) Can. J. Microbiol. 43:1147-1156; Jeng (1990) J. Biol. Chem. 265:3823-3830; U.S. Pat. No. 5,604,118; 5,556,769.

The term “pharmaceutical composition” refers to a composition suitable for pharmaceutical use in a subject. The pharmaceutical compositions of this invention are formulations that comprise a pharmacologically effective amount of a composition comprising a vector or combination of vectors of the invention (i.e., a vector system) and a pharmaceutically acceptable carrier. The invention provides preparations, including pharmaceutical compositions, that are substantially free, or completely free, of helper poxvirus. The term “substantially free of helper virus” or “substantially free of replication competent virus” means that less than about 0.01%, 0.02%, 0.03%, 0.05%, 0.1%, 0.2%, 0.3%, 0.4%, 0.5% or about 1.0% of the capsids in a preparation (e.g., the product of an infection by a vector system of the invention) can replicate in a replication competent cell without some form of complementation by another source, such as the cell, another virus, a plasmid, and the like. In alternative embodiments, pharmaceutical compositions are 100% pure, and about 99.99%, 99.98%, 99.97%, 99.96%, 99.95%, 99.93%, 99.90%, 99.5%, 99.0%, 98%, 97%, 95%, 93% and 90% pure of helper virus.

The term “replication competent cell” or “replication competent host cell” or “producer cell” includes any cell capable of supporting the replication of a poxvirus genome and can support the encapsidation process.

The term “internal ribosomal entry site” or “IRES” refers to all 5′ nontranslated regions that promote “internal” entry of ribosomes independent of the 5′ cap of the mRNA. The IRES is a highly structured RNA secondary structure, such as conserved stem-loop structures. It is an internal ribosomal entry site that mediates cap-independent initiation of translation of viral proteins, a mechanism not found in eukaryotes. It is found in a variety of RNA viruses, including hepatitis C, as described below. See, e.g., Jang (1990) Enzyme 44:292-309; Honda (1999) J. Virol. 73:1165-1174; Psaridi (1999) FEBS Lett. 453:49-53; and, U.S. Pat. Nos: 6,193,980; 6,096,505; 5,928,888; 5,738,985.

The term “ribozyme” describes a self-cleaving DNA sequence, many of which are well known in the art, as are means to isolate, clone and manipulate ribozyme sequences, see, e.g., U.S. Pat. Nos. 6,210,931; 6,043,077; 6,143,503; 6,130,092; 6,087,484; 6,069,007; 5,912,149; 5,773,260; 5,631,115.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogues of natural nucleotides. The term also encompasses nucleic-acid-like structures with synthetic backbones, see e.g., Oligonucleotides and Analogues, a Practical Approach, ed. F. Eckstein, Oxford Univ. Press (1991); Antisense Strategies, Annals of the N.Y. Academy of Sciences, Vol 600, Eds. Baserga et al. (NYAS 1992); Milligan (1993) J. Med. Chem. 36:1923-1937; Antisense Research and Applications (1993, CRC Press), WO 97/03211; WO 96/39154; Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of polypeptide coding sequences in the vectors of invention.

### General Techniques

The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to mammalian cells, e.g., bacterial, yeast, insect or plant systems.

Alternatively, these nucleic acids can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with an appropriate primer sequence.

Techniques for the manipulation of nucleic acids, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

### RNA Viruses

Hepatitis C Virus

The invention provides novel recombinant hepatitis C virus (HCV) genomic sequences, viral particles containing these HCV sequences and methods for making them. Hepatitis C virus (HCV) is a positive-stranded RNA virus. Its genome consists of a single RNA molecule. It contains a 5′ untranslated region (UTR), a polyprotein-coding region and a 3′ UTR. An internal ribosome entry site (IRES) is present in the 5′ UTR (Houghton, Fields Virology, supra, p1035-1058). One hepatitis virus IRES has been described as a 341-nucleotide 5′ non-translated region that is the most conserved part of the hepatitis C virus (HCV) genome. See, e.g., Kolupaeva (2000) J. Virol. 74:6242-6250; Hellen (1999) J. Viral Hepatology 6:79-87. The sequences of the full length genomic HCV RNA for several strains are available; see, e.g., Choo, et al., Science 244: 359; Aizaki et al., Hepatology 27:621-627; Tanaka (1995) Biochem. Biophys. Res. Comm. 215: 744.

HCV is one of the RNA viruses that have not been successfully grown in cell culture. It has been reported that HCV obtained from the infected patients is able to infect human primary hepatocytes (Carloni (1993) Archives of Virology 8:31-39; lacovacci (1993) Research in Virology 144:275-279; Fournier (1998) J. Gen. Virol. 79: 2367-2374), peripheral blood mononuclear cells (Bouffard (1992) J. Infectious Diseases 166: 1276-1280) as well as some cell lines such as human T cell line HPBMa10-2, B cell line Daudi (Bertolini (1993) Research in Virology 144: 281-285; Shimizu (1992) Proc. Natl. Acad. Sci. USA 89:5477-5481) and hepatocyte cell lines (Tagawa (1995) J. Gastroenterol. Hepatol. 10:523-527; Seipp (1997) J. Gen. Virol. 78: 2467-2476; Yoo (1995) J. Virol. 69:32-38). However, the replication of HCV in these cells is generally transient and very inefficient. Another approach to produce HCV in cell culture is by transfecting human hepatoma cell line Huh7 with HCV genomic RNA synthesized by in vitro “run-off” transcription (see, e.g., Yoo (1995) J. Virology 69:32-38). Although it was reported that infectious viral particles were produced from the transfected cells, the replication efficiency of this method is very poor.

Methods for generating and manipulating recombinant RNA viral genomic sequences and vectors, including hepatitis genomes and viruses, e.g., hepatitis C, are well known in the art, see, e.g., U.S. Pat. Nos. 6,156,495; 6,153,421; 6,110,465; 5,981,274; 5,849,532; 5,789,559.

Rhinoviruses

The invention provides novel recombinant rhinovirus genomic sequences, viral particles containing these rhinovirus sequences and methods for making them.

Rhinovirus is a positive-stranded RNA virus. Its genome consists of a single-strand RNA molecule. It contains a 5′ UTR, a polyprotein-coding region and a 3′ UTR with poly(A) at the 3′ terminus, a small protein (VPg is attached to the 5′ end of the genome) . The sequence of the full length genomic RNA has been published (see, e.g., Callahan (1985) Proc. Natl. Acad. Sci. USA 82:732-736). Rhinoviruses can grow in human and some primate cells. The most commonly used human cell lines for rhinovirus growth are the WI-38 line of diploid fibroblasts (Hayflick (1961) Exp. Cell. Res. 25: 585-621), the fetal tonsil line (Fox (1975) Am. J. Epidemiol. 101: 122-143), the MRC-5 line (Jacobs (1970) Nature 227:168-170) and HeLa cell line (Conant (1968) J. Immunol. 100: 107-113). Although there is no report on generation of rhinovirus from the cloned genomic RNA, it has been demonstrated that infectious poliovirus was generated from transfection of human cells with the in vitro-transcribed viral RNA (Semler (1984) Nucleic Acids Res.12: 5123-5141). Poliovirus belongs to the picomaviridae family, as does rhinovirus.

Methods for generating and manipulating recombinant RNA viral genomic sequences and vectors, including picomaviridae genomes and viruses, e.g., rhinovirus and poliovirus, are well known in the art, see, e.g., McKnight (1998) RNA 4:1569-1584, and U.S. Pat. Nos. 6,156,538; 5,614,413; 5,691,134; 5,753,521; 5,674,729.

Influenza Viruses

The invention provides novel recombinant influenza genomic sequences, viral particles containing these influenza sequences and methods for making them.

Influenza virus is a negative-stranded RNA virus. Its genome consists of segmented single-stranded RNA molecules. Influenza A and B viruses each contain eight segments, and influenza C viruses contain seven segments (see, e.g., Lamb et al., 1996, Fields Virology, supra). The complete sequences of influenza A, B, and C viruses are available. Influenza viruses can grow in embryonated eggs and kidney cells. Generation of the viruses from the cloned cDNA of the genomic RNA molecules was reported by, e.g., Neumann (1999) Proc. Natl. Acad. Sci. USA 96:9345-9350; Hoffmann (2000) Virology 267:310-317. The reported system employs human RNA polymerase to synthesize both the viral RNA and mRNA in human embryonic kidney cells 293T and results in production of influenza virions.

Methods for generating and manipulating recombinant RNA viral genomic sequences and vectors, including influenza genomes and viruses, are well known in the art, see, e.g., Kemdirim (1986) Virology 152:126-135; and U.S. Pat. Nos. 5,837,852; 5,879,925.

Lentivirus-derived Vectors

The invention provides novel recombinant lentivirus genomic sequences, viral particles containing these lentivirus sequences and methods for making them.

Lentivirus-derived vectors and the related packaging systems were initially created by Naldini (1996) Science 272: 263-267, and recently improved by Dull et al. to further reduce the potential of generating replication competent HIV (Dull (1998) J. Virol. 72:8463-71). In this system, four plasmids which separately encode HIV Gag-Pol, Rev, vesicular stomatitis virus G (VSV-G) envelope protein and the vector RNA are used to transfect human kidney epithelial cell line 293 T. After the HIV-1 precursor polyproteins Gag/Pol and Gag are synthesized in the vector particle-producing cells, they will in turn package the vector RNA and bud from the plasma membrane to form viral particles. When VSV-G protein is co-expressed with Gag-Pol, the resulting viral particles have VSV-G protein being displayed on their surface, which will facilitate entry of the particles into host cells.

Methods for generating and manipulating recombinant RNA viral genomic sequences and vectors, including lentivirus genomes and viruses, are well known in the art, see, e.g., Kirchhoff (1990) Virology 177:305-311; and U.S. Pat. Nos. 6,165,782; 5,994,516; 5,994,136; 5,747,324; 5,624,795; 5,614,404.

Poxviruses

The invention provides methods for producing an encapsidated RNA virus and RNA genomic sequences comprising use of replication defective poxviruses. In one aspect, coding sequence for a bacteriophage polymerases are cloned into the replication defective poxviruses such that the coding sequences are operably linked to a poxvirus promoter.

Poxvirus is a DNA virus. It uses its own enzymes to carry out DNA replication and transcription. The replication of the virus is carried out entirely in the cytoplasm of host cells (see, e.g., Moss, Fields Virology, supra, p. 2673-2702). The vaccinia DNA polymerase can also replicate plasmids that are present in the cytoplasm to produce heterogeneous and large linear DNA (Moss, Fields Virology, supra, p. 2673-2702). If the DNA contains vaccinia promoters, it can be transcribed by the vaccinia RNA polymerase. Because of these properties, poxviruses have been widely used for expression of foreign proteins (see, e.g., Panicali and Paoletti, 1982, Proc. Natl. Acad. Sci. USA 79: 4927-31; Hackett et al., 1982, Proc. Natl. Acad. Sci. USA 79: 7415-19; Scheiflinger et al., 1992, Proc. Natl. Acad. Sci. USA 89:9977-81; Merchlinsky and Moss, 1992, Virol. 190: 522-26). One of the vaccinia expression systems employs bacteriophage RNA polymerase, for example, T7, T3 or SP6 (see, e.g., Fuerst et al., 1987, Mol. Cell. Biol. 7:2538-2544; Rodriguez et al., 1990, J. Viol. 64: 4851-4857; Usdin et al., 1993, BioTech. 14: 222-224). In this system, the recombinant vaccinia virus encoding bacteriophage RNA polymerase is used for in vivo transcription. DNA to be transcribed is cloned into a plasmid downstream of a bacteriophage promoter. Cells are infected with the recombinant vaccinia virus and then transfected with the plasmid. Bacteriophage RNA polymerase will be synthesized upon vaccinia infection and subsequently transcribe the DNA downstream of a bacteriophage promoter.

Poxvirus can be rendered non-viable by suppressing the expression of one or more of its essential genes. One method is to insert an inducible promoter in front of the open reading frame (ORF) of an essential gene (see, e.g., Fuerst et al., 1989, Proc. Natl. Acad. Sci. USA 86:2549-2553). For example, a conditional lethal, inducer-dependent vaccinia virus contains a inducible D13L gene (see, e.g., Zhang (1992) Virol. 187: 643-653). In the absence of inducer, the expression of the essential gene is inhibited. Another method is to delete an essential gene from the virus genome. Falkner et al. developed a method using complementing cell lines that stably express the corresponding essential protein to propagate defective vaccinia recombinants that lacks an essential gene. (Falkner et al. (1998) U.S. Pat. No. 5,770,212, U.S. Pat. No. 5,766,882).

Methods for generating and manipulating recombinant RNA viral genomic sequences and vectors, including poxvirus, e.g., vaccinia, are well known in the art, see, e.g., U.S. Pat. Nos. 6,214,353; 6,168,943; 6,130,066; 6,051,410; 5,990,091; 5,849,304; 5,770,212; 5,770,210; 5,766,882; 5,762,938; 5,747,324; 5,718,902; 5,605,692.

Formulation and Administration Pharmaceuticals

The invention also provides vectors formulated as pharmaceuticals for the transfer of nucleic acids into cells in vitro or in vivo. The vectors, vector systems and methods of the invention can be used to produce replication defective gene transfer and gene therapy vectors, particularly to transfer nucleic acids to human cells in vivo and in vitro. Using the vector system and methods of the invention, these sequences can be packaged as gene therapy vector preparations that are substantially free of helper virus and used as pharmaceuticals in, e.g., gene replacement therapy (in somatic cells or germ tissues) or cancer treatment; see, e.g., Karpati (1999) Muscle Nerve 16:1141-1153; Crystal (1999) Cancer Chemother. Pharmacol. 43 Suppl:S90-9.

The vectors, vector systems, pharmaceutical compositions and methods of the invention can also be used in non-human systems. For example, the vectors of the invention can be used in gene delivery in laboratory animals (e.g., mice, rats) as well as economically important animals (e.g., swine, cattle); see, e.g., Mayr (1999) Virology 263:496-506; Mittal (1996) Virology 222:299-309; Prevec (1990) J. Infect. Dis. 161:27-30.

These pharmaceuticals can be administered by any means in any appropriate formulation. Routine means to determine drug regimens and formulations to practice the methods of the invention are well described in the patent and scientific literature, and some illustrative examples are set forth below. For example, details on techniques for formulation, dosages, administration and the like are well described in the scientific and patent literature, see, e.g., the latest edition of Remington's Pharmaceutical Sciences, Maack Publishing Co, Easton Pa.

### Pharmaceutical Compositions

The invention provides a replication defective adenovirus preparation substantially free of helper virus with a pharmaceutically acceptable carrier (excipient) to form a pharmacological composition. The pharmaceutical composition of the invention can further comprise other active agents, including other recombinant viruses, plasmids, naked DNA or pharmaceuticals (e.g., anticancer agents).

Pharmaceutically acceptable carriers can contain a physiologically acceptable compound that acts, e.g., to stabilize the composition or to increase or decrease the absorption of the agent and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipients or other stabilizers and/or buffers. Detergents can also used to stabilize the composition or to increase or decrease the absorption of the pharmaceutical composition (see infra for exemplary detergents).

Other physiologically acceptable compounds include wetting agents, emulsifying agents, dispersing agents or preservatives that are particularly useful for preventing the growth or action of microorganisms. Various preservatives are well known, e.g., ascorbic acid. One skilled in the art would appreciate that the choice of a pharmaceutically acceptable carrier, including a physiologically acceptable compound depends, e.g., on the route of administration of the adenoviral preparation and on the particular physio-chemical characteristics of any co-administered agent.

The compositions for administration will commonly comprise a buffered solution comprising adenovirus in a pharmaceutically acceptable carrier, e.g., an aqueous carrier. A variety of carriers can be used, e.g., buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of capsids in these formulations can vary widely, and will be selected primarily based on fluid volumes, viscosities, body weight and the like in accordance with the particular mode of administration selected and the patient's needs.

### Determining Dosing Regimens

The pharmaceutical formulations of the invention can be administered in a variety of unit dosage forms, depending upon the particular condition or disease, the general medical condition of each patient, the method of administration, and the like. In one embodiment, the concentration of capsids in the pharmaceutically acceptable excipient is between about 10³ to about 10¹⁸ or between about 10⁵ to about 10¹⁵ or between about 10⁶ to about 10¹³ particles per mL in an aqueous solution. Details on dosages are well described in the scientific and patent literature, see, e.g., the latest edition of Remington's Pharmaceutical Sciences; Sterman (1998) Hum. Gene Ther. 9:1083-1092; Smith (1997) Hum. Gene Ther. 8:943-954.

The exact amount and concentration of RNA virus and the amount of formulation in a given dose, or the “therapeutically effective dose” is determined by the clinician, as discussed above. The dosage schedule, i.e., the “dosing regimen,” will depend upon a variety of factors, e.g., the stage and severity of the disease or condition to be treated by the gene therapy vector, and the general state of the patient's health, physical status, age and the like. The state of the art allows the clinician to determine the dosage regimen for each individual patient and, if appropriate, concurrent disease or condition treated. Genetically engineered RNA vectors have been used in gene therapy, see, e.g., Bosch (2000) Hum. Gene Ther. 11:1139-1150; Mukhtar (2000) Hum. Gene Ther. 11:347-359; Deglon (2000) Hum. Gene Ther. 11:179-190; Sallberg (1998) Hum. Gene Ther. 9:1719-1729. These illustrative examples can also be used as guidance to determine routes of administration, formulations, the dosage regiment, i.e., dose schedule and dosage levels administered when practicing the methods of the invention.

Single or multiple intrathecal administrations of RNA virus formulation can be administered, depending on the dosage and frequency as required and tolerated by the patient. Thus, one typical dosage for regional (e.g., IP or intrathecal) administration is between about 0.5 to about 50 mL of a formulation with about 10¹³ viral particles per mL. In an alternative embodiment, dosages are from about 5 mL to about 20 mL are used of a formulation with about 10⁹ viral particles per mL. Lower dosages can be used, such as is between about 1 mL to about 5 mL of a formulation with about 10⁶ viral particles per mL. Based on objective and subjective criteria, as discussed herein, any dosage can be used as required and tolerated by the patient.

The exact concentration of virus, the amount of formulation, and the frequency of administration can also be adjusted depending on the levels of in vivo (e.g., in situ) transgene expression and vector retention after an initial administration.

### Routes of Delivery

The pharmaceutical compositions of the invention, comprising the RNA virus constructs of the invention, can be delivered by any means known in the art systemically (e.g., intravenously), regionally, or locally (e.g., intra- or peri-tumoral or intracystic injection, e.g., to treat bladder cancer) by, e.g., intraarterial, intratumoral, intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa), intra-tumoral (e.g., transdermal application or local injection). For example, intra-arterial injections can be used to have a “regional effect,” e.g., to focus on a specific organ (e.g., brain, liver, spleen, lungs). For example, intra-hepatic artery injection can be used if the anti-tumor regional effect is desired in the liver; or, intra-carotid artery injection. If it is desired to deliver the viral preparation to the brain, (e.g., for treatment of brain tumors), it is injected into a carotid artery or an artery of the carotid system of arteries (e.g., occipital artery, auricular artery, temporal artery, cerebral artery, maxillary artery, etc.).

The vectors of the present invention, alone or in combination with other suitable components can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations such as in a nebulizer or an atomizer. Typically such administration is in an aqueous pharmacologically acceptable buffer as described above. Delivery to the lung can be also accomplished, e.g., by use of a bronchoscope. Gene therapy to the lung includes, e.g., gene replacement therapy for cystic fibrosis (using the cystic fibrosis transmembrane regulator gene) or for treatment of lung cancers or other respiratory conditions.

Additionally, the vectors employed in the present invention may be made into suppositories by mixing with a variety of bases such as emulsifying bases or water-soluble bases. Formulations suitable for vaginal administration may be presented as pessaries, tampons, creams, gels, pastes, foams, or spray formulas.

The pharmaceutical formulations of the invention can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The constructs of the invention can also be administered in a lipid formulation, more particularly either complexed with liposomes to for lipid/nucleic acid complexes (e.g., as described by Debs and Zhu (1993) WO 93/24640; Mannino (1988) supra; Rose, U.S. Pat. No. 5,279,833; Brigham (1991) WO 91/06309; and Felgner (1987) supra) or encapsulated in liposomes, as in immunoliposomes directed to specific tumor markers. It will be appreciated that such lipid formulations can also be administered topically, systemically, or delivered via aerosol.

Kits

The invention provides kits that contain the vectors, vector systems or pharmaceutical compositions of the invention. The kits can also contain replication-competent cells. The kit can contain instructional material teaching methodologies, e.g., means to isolate replication defective RNA viruses. Kits containing pharmaceutical preparations can include directions as to indications, dosages, routes and methods of administration, and the like.

Without further elaboration, it is believed that one skilled in the art can, using the preceding description, utilize the present invention to its fullest extent. The following examples are to be considered illustrative and thus are not limiting of the remainder of the disclosure in any way whatsoever.

### EXAMPLES

The following example is offered to illustrate, but not to limit the claimed invention.

### Example 1

### Production of HCV-ribozyme-T7 Terminator-poly(a) RNA Viral Particles

The following example provides an exemplary method of the invention for producing infectious HCV viral particles, in particular, HCV-ribozyme-T7 terminator-poly(a) RNA viral particles.

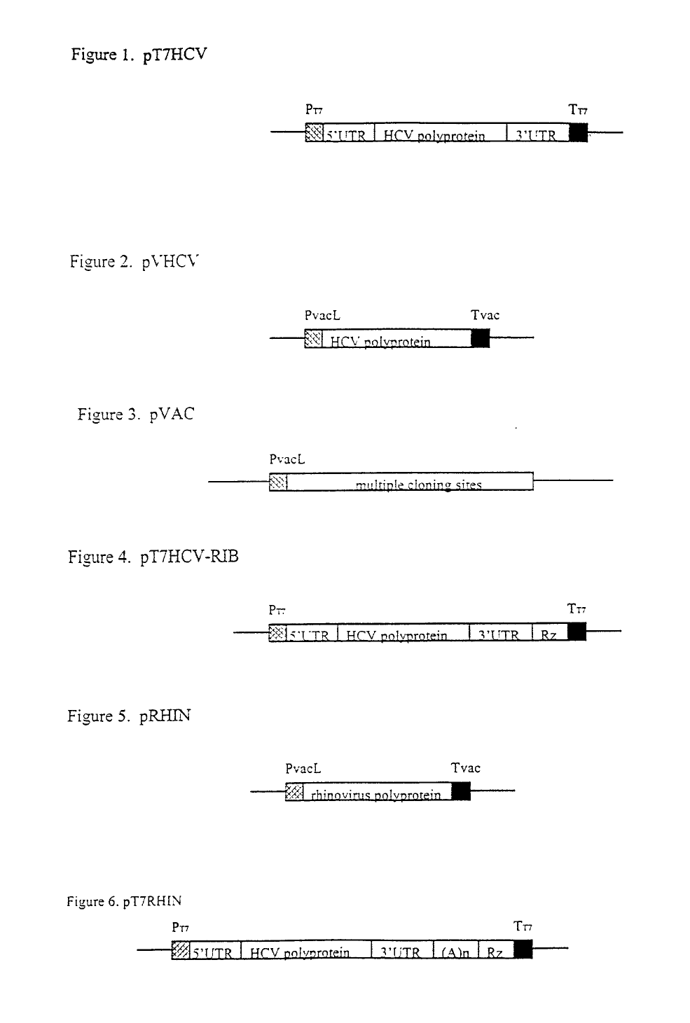

The first step to produce infectious HCV viral particles was to construct two plasmids: pT7HCV (FIG. 1) which contains a DNA copy of a full length HCV genomic RNA and pVHCV (FIG. 2) which contains the HCV polyprotein-coding region downstream of a synthetic vaccinia late promoter. In the pT7HCV plasmid, a DNA copy of the HCV genome, which includes 5′ UTR, the open reading frame (ORF) of the polyprotein and 3′ UTR, is flanked by a bacteriophage T7 promoter (PT7) and a bacteriophage T7 terminator (TT7). The thin lines in FIG. 1 represent the pUC19 backbone. In the plasmid pVHCV, the HCV polyprotein-coding region is linked to a vaccinia later promoter (PvacL). The thin lines in FIG. 2 represent the pUC19 backbone.

Based on the sequence of HCV genome reported by Aizaki (1998) Hepatology 27:621-627, a DNA copy of HCV was generated from the serum of a HCV infected patient by reverse transcription coupled polymerase chain reaction (RT-PCR) and cloned into the pUC 19 plasmid. To construct pT7HCV, the cDNA encoding the HCV genomic RNA was amplified using the T7 promoter- and terminator-tagged primers. The primers have the following sequence:

| | |
|---|---|
| 5′-TAATACGACTCACTATAGGGCCAGCCCCCTGATGGGGGCGACACTCC-3′ | (SEQ ID NO:1) |
| 5′-CAAAAAACCCCTCAAGACCCGTTTAGAGGCCCCAAGGGGTTATGCTAGACATGATCTGCAGAGAGGCCAGTATCAG-3′ | (SEQ ID NO:2) |

The PCR product was then inserted into pUC19 (Life Technology), resulting in pT7HCV. To make pVHCV, a DNA copy of the HCV polyprotein-coding region was generated by RTPCR and cloned into plasmid pVAC (FIG. 3) downstream of a vaccinia late promoter (described by Moss (1996) Fields Virology, supra, p. 2673-2702), resulting in pVHCV. In this construct, the 5′ untranslated region is deleted to facilitate the cap-dependent translation. In the pVAC plasmid, the multiple cloning site region is linked to a vaccinia late promoter (PvacL). The thin line in FIG. 3 represents the pUC19 backbone.

Next, HeLa cells (10⁶ cells) in a T25 flask were co-transfected with 10 μg of pT7HCV and 10 μg of pVHCV in 2 ml of MEM containing 2.5% fetal bovine serum using DOTAP (Boehringer Mannheim) for transfection. Four hours after transfection, the medium was removed, and the cells were inoculated with 10⁷ pfu of vT7ΔD13L in MEM containing 2.5% fetal bovine serum. After two hours, the inoculum was removed and the cells were cultured in MEM containing 10% fetal bovine serum. After incubating at 30° C., 5% CO₂ for 48 hours, the cell culture media that contained HCV virions was collected.

To determine the infectious titer of the HCV preparation, a series of 10 fold dilution of the collected cell culture supernatant was made with OPTI-MEM™ (GIBCOLBRL) containing 1% fetal bovine serum and 1 ml of the diluted supernatant was added to each well of a 12-well cell culture plate. In each well, 5×10⁵ Huh7 cells were seeded on the previous day. After 24 hours, the inoculum was removed and replaced with 1 ml of fresh DMEM containing 10% fetal bovine serum. After being cultured for 1-2 days, the cells were collected and total RNA was extracted from the cells. The negative strand HCV RNA was detected using RT-PCR to amplify the 300 bp fragment of HCV 5′ untranslated region. The primer used for reverse transcription has the following sequence:

| | |
|---|---|
| 5′-ATGATGCACGGTCTACGAGACCTCCCGGGGC-3′ | (SEQ ID No.3) |
| The primers used for PCR had the | |
| following sequences: | |
| 5′-CCAGCCCCCTGATGGGGGCGACA-3′ | (SQE ID No.4) |
| 5′-ACTCGCAAGCACCCTATCAGGCA-3′ | (SQE ID No.5) |

The HCV virion that contains HCV genomic RNA without the T7 terminator sequence and poly(A) was also generated as the following. First, a T7 primer-tagged primer (SEQ ID. 2) and a T7 terminator-tagged primer which contains restriction sites Mfe 1 and Pac 1 were used to amplify the DNA copy of HCV genomic RNA. The T7 terminator-tagged primer has the following sequence:

| | |
|---|---|
| 5′-CAAAAAACCCCTCAAGACCCGTTTAGAGGCCCCAAGGGGTTAT | |
| GCTACAATTGCCCCTTAATTAAGACACACATGATCTGCAGAGAGGCCAGTATCAG-3′ | (SEQ ID No.6). |

The underlined shows the Mfe 1 and Pac 1 sites. The PCR product was inserted into pUC19. The resulting plasmid was then digested with Mfe 1 and Pac 1 and ligated with a hairpin-ribozyme cDNA resulting in pT7HCV-RIB (FIG. 4). In the pT7HCV-RIB plasmid, a DNA copy of the HCV genomic RNA and the adjacent hairpin ribozyme (Rz) is flanked by a bacteriophage T7 promoter (PT7) and a bacteriophage T7 terminator (TT7). The thin lines in FIG. 4 represent the pBR322 backbone.

The cDNA was formed by hybridization of following two oligos:

| | |
|---|---|
| 5′-TCCTCCAATTAAAGAACACAACCAGAGAAACACACGTTGTGGTATATTACCTGGTAC-3′ | (SEQ ID No.7) |
| 5′-AATTGTACCAGGTAATATACCACAACGTGTGTTTCTCTGGTTGTGTTCTTTAATTGGAGGAAT-3′ | (SEQ ID No.8). |

The cells were co-transfected with pT7HCV-RIB and pVHCV. The transfected cell were then infected with the helper vaccinia recombinant vT7ΔD13L. In this helper vaccinia recombinant, the D13L is deleted according to the method provided by Falkner, et al., U.S. Pat. No. 5,770,212. After the HCV-ribozyme-T7 terminator-poly(a) RNA is synthesized, it was cleaved to generate HCV RNA with only two extra nucleotides GT and a 2′,3′cyclic phosphate at the 3′ end. The resulting virions had a slightly higher infectivity than that contains the HCV RNA tailed with a T7 terminator and poly(A).

### Example 2

### Production of Infectious Rhinovirus Particles

The following example provides an exemplary method of the invention for producing infectious rhinovirus viral particles.

Production of rhinovirus was carried out by plasmid transfection and helper vaccinia infection. Using the method described here, a high infectious titer viral stock was obtained in a few days. Based on the published the genomic sequence of human rhinovirus 14 (Stanway et al., 1984, Nucleic Acids Res. 12: 7859-7875), a cDNA copy of the complete rhinovirus genome including a 70 nucleotides long ploy(A) tract was generated by RTPCR. The cDNA was then cloned into the pBR322 plasmid. From the cloned rhinovirus cDNA, two plasmids used for the virus production were constructed.

A pUC19-based plasmid, pRHIN (FIG. 5), is used for the expression of the viral protein of rhinovirus. It contains the ORF of the viral polyprotein downstream of a vaccinia later promoter.

Another plasmid, pT7RHRIN (FIG. 6), is used as a template for the synthesis of rhinovirus genomic RNA. For construction of pT7RIHN, a T7 promoter-tagged primer and a T7 terminator-tagged primer which contains restriction sites Mlu1 and Pac 1 were used to amplify the cDNAs that encode the rhinovirus genomic RNA. The T7 promoter-tagged primer and T7 terminator-tagged primer has the following sequence:

| | |
|---|---|
| 5′-TAATACGACTCACTATAGGTTAAAACTGGGTGTGGGTTGTTCCCAC-3′ | (SEQ ID No.9) |
| 5′CAAAAAACCCCTCAAGACCCGTTTAGAGGCCCCAAGGGGTTATGCTAA | (SEQ ID No.10) |
| CGCGTCCCCTTAATTAAGACACTTTTTTTTTTTTTTTTTTTTTTTTTTTTTT-3′ | |

The underlined shows the Mlu 1 and Pac 1 sites. The PCR product was inserted into pUC19. The resulting plasmid was then digested with Mlu 1 and Pac 1 and then ligated with a hairpin-ribozyme cDNA. The hairpin-ribozyme cDNA was formed by hybridization of following two oligos:

| | |
|---|---|
| 5′-TCCTCCAATTAAAGAACTTTACCAGAGAAACACACGTTGTGGTATATTACCTGGTA-3′ | (SEQ ID No.11) |
| 5′-CGCGTACCAGGTAATATACCACAACGTGTGTTTCTCTGGTAAAGTTCTTTAATTGGAGGAAT-3′ | (SEQ ID NO.12). |

The resulting pT7RHIN contains the cDNA of the viral genomic RNA (including a polyA tract) linked to a hairpin-ribozyme cDNA. The rhinovirus-ribozyme-coding sequence is flanked by the T7 promoter and T7 terminator.

For production of rhinovirus, HeLa cells (10⁶ cells) in a T25 flask were transfected with 10 μg pRHIN and 10 μg pT7RHIN using DOTAP (Boehringer Mannheim) followed by vT7ΔD13L infection. The infection was allowed to proceed for 2 hours. Then inoculum was removed and replaced with fresh MEM containing 2.5% fetal bovine serum. After incubation at 30° C. for 48 hours, supernatant that contained rhino virions was collected. To determine the infectious titer of the rhinovirus preparation, a series of 10 fold dilution of the cell culture supernatant was made with DMEM containing 10% fetal bovine serum. Then 1 ml of the diluted viruses was added to each well of a 12 well cell culture plate. In each well, 10⁶ HeLa cells were seeded on the previous day. After incubation at 37° C. for 2-3 days, the number of plaques was counted.

In comparison to the natural rhinovirus RNA, the virion RNA generated by this method contains two extra nucleotides and a 2′,3′ cyclic phosphate at the 3′ terminus.

### Example 3

### Production of Infectious Influenza A Viral Particles

The following example provides an exemplary method of the invention for producing infectious influenza A viral particles.

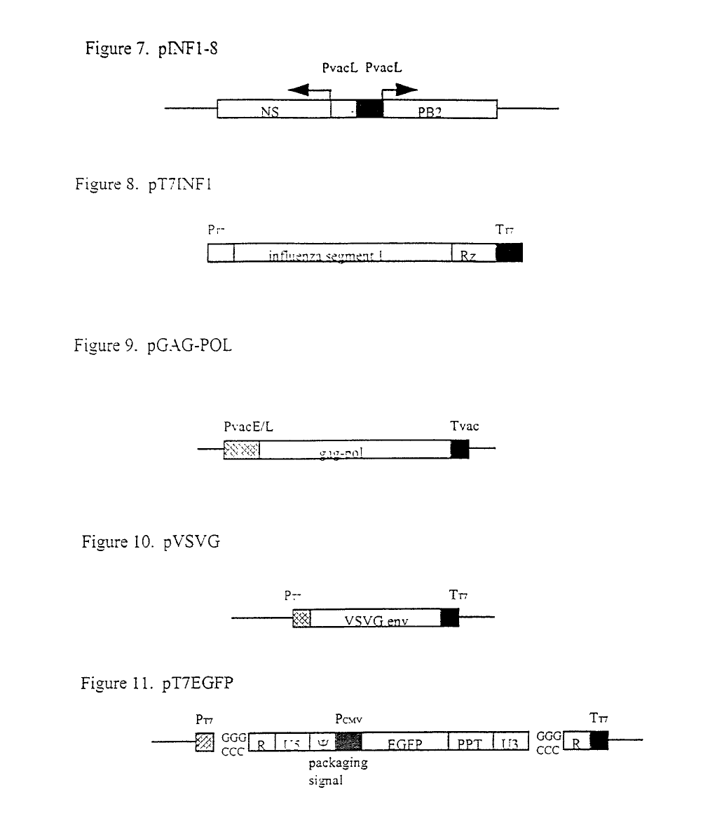

Production of influenza A was carried out by plasmid transfection followed by helper vaccinia infection. A high infectious titer viral stock was obtained in a few days. Using the published the sequences of the RNA segments of human influenza virus A/PR/8/34 (see, e.g., Fields et al., 1982, Cell 28:303-313; Fields et al., 1981, Nature 290: 213-217; Winter et al., 1982, Nucleic Acids Res. 10: 2135-2143; Winter et al., 1981, Nature 292: 72-75; Winter et al., 1981, Virology 114: 423-428; Winter et al., 1981, Nucleic Acids Res. 8: 1965-1974; Baez et al., 1980, Nucleic Acids Res. 8: 5845-5858), primers were designed and the cDNA copies of the eight RNA segments were generated by RT-PCR. The cDNAs were then cloned into the pUC19 plasmids individually. From the cloned cDNAs, two types of plasmids used for the virus production were constructed. One is for the expression of the viral proteins. Four plasmids pINF1-8, pINF2-7, pINF3-6, and pINF4-5 were constructed. Each carries two viral protein expression cassettes under the control of vaccinia late promoters (FIG. 7).

pINF1-8 contains the ORFs of PB2 and NS, pINF2-7 contains PB1 and M, pINF 3-6 contains PA and NA, and pINF4-5 contains HA and NP. The other type of plasmids is for the expression of the genomic RNA segments. Eight plasmids pT7INF1, pT7INF2, pT7INF3, pT7INF4, pT7INF5, pT7INF6, pT7INF7, and pT7INF8 were constructed on the base of pUC19. Each plasmid carries one transcription unit for one of the eight genomic RNA segments. Within the transcription unit, the cDNA encoding the genomic RNA is placed between a T7 promoter and a T7 terminator in such an orientation that transcription of the cDNA by T7 RNA polymerase will generate the genomic (negative strand) RNA. A hairpin-ribozyme cDNA is inserted between the cDNA and the T7 terminator (FIG. 8).

For construction of plasmids pT7INF1, pT7INF2, pT7INF3, pT7INF4, pT7INF5, pT7INF6, pT7INF7, and pT7INF8, eight pairs of T7 promoter-tagged primers and T7 terminator-tagged primers which contain the restriction sites Mlu 1 and Pac 1 were used to amplify each of the cDNAs which encode the genomic RNA segments. The T7 promoter-tagged primer and T7 terminator-tagged primer for amplification of segment 1 have the following sequences:

| | |
|---|---|
| 5′-TAATACGACTCACTATAGGAGCGAAGCAGGTCAATTATATTCAA-3′ | (SEQ ID No.13) |
| 5′CAAAAAACCCCTCAAGACCCGTTTAGAGGCCCCAAGGGGTTATGCTAC | (SEQ ID No.14) |
| GCGTCCCCTTAATTAAGACACAGTAGAAACAAGGTCGTTTTTAAAC-3′ | |

The underlined shows the Mlu 1 and Pac 1 sites.

The T7 promoter-tagged primer and T7 terminator-tagged primer for amplification of segment 2 have the following sequences:

| | |
|---|---|
| 5′-TAATACGACTCACTATAGGAGCGAAAAGCAGGCAAACCATTTGAATGGAT-3′ | (SEQ ID No.15) |
| 5′-CAAAAAACCCCTCAAGACCCGTTTAGAGGCCCCAAGGGGTTATGCT | (SEQ ID No.16) |
| ACGCGTCCCCTTAATTAAGACACAGTAGGAACAAGGCATTTTTTCATG-3′ | |

The T7 promoter-tagged primer and T7 terminator-tagged primer for amplification of segment 3 have the following sequences:

| | |
|---|---|
| 5′-TAATACGACTCACTATAGGAGCGAAAGCAGGTACTGATCCAAAATGG-3′ | (SEQ ID No.17) |
| 5′-CAAAAAACCCCTCAAGACCCGTTTAGAGGCCCCAAGGGGTTATGCT | (SEQ ID No.18) |
| ACGCGTCCCCTTAATTAAGACACAGTAGAAACAAGGTACTTTTTTG-3′ | |

The T7 promoter-tagged primer and T7 terminator-tagged primer for amplification of segment 4 have the following sequences:

| | |
|---|---|
| 5′-TAATACGACTCACTATAGGAGCGAAAAGCAGGGGAAAATAAAAACAA-3′ | (SEQ ID No.19) |
| 5′-CAAAAAACCCCTCAAGACCCGTTTAGAGGCCCCAAGGGGTTATGCT | (SEQ ID No.20) |
| ACGCGTCCCCTTAATTAAGACACAGTAGAAACAAGGGTGTTTTTCC-3′ | |

The T7 promoter-tagged primer and T7 terminator-tagged primer for amplification of segment 5 have the following sequences:

| | |
|---|---|
| 5′-TAATACGACTCACTATAGGAGCAAAAGCAGGGTAGATAATCACTCACTG-3′ | (SEQ ID No. 21) |
| 5′-CAAAAAACCCCTCAAGACCCGTTTAGAGGCCCCAAGGGGTTATGCT | (SEQ ID No. 22) |
| ACGCGTCCCCTTAATTAAGACACAGTAGAACAAGGGTATTTTTCTTTAATTG-3′ | |

The T7 promoter-tagged primer and T7 terminator-tagged primer for amplification of segment 6 have the following sequences:

| | |
|---|---|
| 5′-TAATACGACTCACTATAGGAGCGAAAGCAGGGGTTTAAAATGAATCC-3′ | (SEQ ID No. 23) |
| 5′-CAAAAAACCCCTCAAGACCCGTTTAGAGGCCCCAAGGGGTTATGCT | (SEQ ID No. 24) |
| ACGCGTCCCCTTAATTAAGACACAGTAGAAACAAGGAGTTTTTTGAAC-3′ | |

The T7 promoter-tagged primer and T7 terminator-tagged primer for amplification of segment 7 have the following sequences:

| | |
|---|---|
| 5′-TAATACGACTCACTATAGGAGCGAAAGCAGGTAGATATTGAAAGATGA-3′ | (SEQ ID No. 25) |
| 5′-CAAAAAACCCCTCAAGACCCGTTTAGAGGCCCCAAGGGGTTATGCT | (SEQ ID No. 26) |
| ACGCGTCCCCTTAATTAAGACACAGTAGAAACAAGGTAGTTTTTTACTCC-3′ | |

The T7 promoter-tagged primer and T7 terminator-tagged primer for amplification of segment 8 have the following sequences:

| | |
|---|---|
| 5′-TAATACGACTCACTATAGGAGCAAAAGCAGGGTGACAAAGACATAATG-3′ | (SEQ ID No. 27) |
| 5′-CAAAAAACCCCTCAAGACCCGTTTAGAGGCCCCAAGGGGTTATGC | (SEQ ID No. 28) |
| TACGCGTCCCCTTAATTAAGACACAGTAGAAACAAGGGTGTTTTTTATTATT-3′ | |

The PCR product was inserted into pUC19. The resulting plasmids were then digested with Mlu 1 and Pac 1 and ligated with a hairpin-ribozyme cDNA. The cDNA was formed by hybridization of following two oligos:

| | |
|---|---|
| 5′-TCCTCCAATTAAAGAACagtACCAGAGAAACACACGTTGTGGTATATTACCTGGTA-3′ | (SEQ ID No. 29) |
| 5′-CGCGTACCAGGTAATATACCACAACGTGTGTTTCTCTGGTactGTTCTTTAATTGGAGGAAT-3′ | (SEQ ID No. 30). |

The resulting pT7INF1, pT7INF2, pT7INF3, pT7INF4, pT7INF5, pT7INF6, pT7INF7, and pT7INF8 each contains the cDNA which encodes the genomic RNA segment one through eight respectively. The cDNA is linked to a hairpin-ribozyme-coding sequence. The resulting cDNA encoding the RNA segment-ribozyme is flanked by a T7 promoter and T7 terminator.

For production of influenza virus, HeLa cells (10⁶ cells) in a T25 flask were co-transfected with 5 μg of each plasmid from pINF1 through pINF8 and 5 μg of each plasmid from pT7INF1 through pT7INF8 using DOTAP (Boehringer Mannheim) followed by vT7ΔD13L infection. The infection was allowed to proceed for 2 hours. Then inoculum was removed and replaced with fresh MEM containing 2.5% fetal bovine serum. After incubation at 30° C. for 48 hours, supernatant that contained influenza A virions was collected. To determine the infectious titer of the virus preparation, a series of 10 fold dilution of the cell culture supernatant was made with DMEM containing 10% fetal bovine serum. Then 1 ml of the diluted viruses was added to each well of a 12 well cell culture plate. In each well, 10⁶ MDCK (Madin-Darby canine kidney) cells were seeded on the previous day. After incubation, the number of plaques was counted.

In comparison to the natural influenza virus RNA, the virion RNA segments generated by this method contain two extra nucleotides and 2′,3′ hydroxyl phosphate at the 3′ terminus.

### Example 4

### Production of HIV-1-derived Vector Particles

The following example provides an exemplary method of the invention for producing HIV-1-derived vector particles.

Based on the sequences of HIV-1 strain HXB2 (Wong-Staal et al. (1985) Nature 313: 277-284) and vesicular stomatitis virus G glycoprotein (VSV-G) (Rose and Bergmann (1983) Cell 34: 513-524), three plasmids were constructed for the vector production. pGAG-POL (FIG. 9) which was used for the expression of HIV-1 HXB2 gag-pol contains the coding region of gag-pol cloned between a vaccinia 7.5 early/later promoter and a vaccinia terminator. Another plasmid pVSV-G (FIG. 10) contains the VSV-G-coding region cloned into the pT7 plasmid between a T7 promoter and a T7 terminator (Rose and Bergmann (1983) Cell 34: 513-524). Since in vaccinia virus-infected cells, only 10% of the transcripts synthesized in the cytoplasm by T7 RNA polymerase are capped and thus can be translated, utilization of T7 RNA polymerase for the expression of VSV-G envelope glycoprotein can avoid excessive envelope glycoprotein on the cell surface. Over-expression of VSV-G can causes massive cell-cell fusion and toxicity in the cells. These effects will reduce the yield of the vector particles. The third plasmid pT7EGFP (FIG. 11) was used as the template for synthesis of the vector RNA molecule. This RNA molecule has the HIV-1 5′ LTR followed by the packaging signal sequence and a CMV promoter-controlled transcription unit for the enhanced green fluorescence protein followed by a polypurine tract sequence and the 3′ LTR. Since there is a triple G between 3′ U3 and 3′ R to allow base pairing with the triple C at the 3′ terminal of the strong stop DNA during reverse transcription, no insertion of a triple G is needed. A DNA copy of such the vector RNA molecule was cloned between a T7 promoter and a T7 terminator resulting in pT7EGFP.

For production of HIV-1-derived vector particles, HeLa cells (10⁶ cells) in a T25 flask were co-transfected with 10 μg pGAG-POL, 10 μg pVSVG and 10 μg pT7EGFP in 4 ml of MEM containing 2.5% fetal bovine serum using DOTAP (Boehringer Mannheim) for transfection. 4 hours after transfection, 10⁷ pfu of purified helper vaccinia recombinant vT7ΔD13L were added to the transfection medium. The inoculum was removed two hours after inoculation and replaced with fresh DMEM containing 10% fetal bovine serum. The cells were cultured for 48 hours and then the cell culture supernatant containing the viral vectors is collected. To titer the vectors, a series of 10 fold dilution of the supernatant is made and then 1 ml of the diluted vectors was added to each well of a 12-well cell culture plate. In each well, 10⁵ HeLa cells were seeded on the previous day. After 24 hours, the green fluorescent cells were counted using a fluorescent microscope.

| | |
|---|---|
| SEQUENCE ID LIST | |
| 5′-AAAAATTGAAATTTTATTTTTTTTTTTGGAATATAAATA-3′ | (SEQ ID No. 1) |
| 5′-CATAGTATCGATTACACCTCTACCG-3′ | (SEQ ID No. 2) |
| 5′-GAGAGGTTTTCTACTTGCTCATTAG-3′ | (SEQ ID No. 3) |
| 5′-AAAAGTAGAAAAAATAATTTTTTTTTTGAGATTTAAATA-3′ | (SEQ ID. No. 4) |
| 5′-TTAATTGTTGTCGCCCATAATCTTGGTAATACTTACCCC-3′ | (SEQ ID No.5) |
| 5′-ATGAATAATACTATCATTAATTCTTTG-3′ | (SEQ ID No.6) |
| 5′-TTTTTTTTTTTTTTTTTTAGGATTTAAATA-3′ | (SEQ ID No. 7) |
| 5′-TAATACGACTCACTATAGGGCCAGCCCCCTGATGGGGGCGACACTCC-3′ | (SEQ ID No. 8) |
| 5′-CAAAAAACCCCTCAAGACCCGTTTAGAGGCCCCAAGGGGTTATGCTAGACATGATCTGCAGAGAGGCCAGTATCAG-3′ | (SEQ ID No. 9) |
| 5′-ATGATGCACGGTCTACGAGACCTCCCGGGGC-3′ | (SEQ ID No. 10) |
| 5′-CCAGCCCCCTGATGGGGGCGACA-3′ | (SQE ID No. 11) |
| 5′-ACTCGCAAGCACCCTATCAGGCA-3′ | (SQE ID No. 12) |
| 5′-GCGCCAGTCCTCCGATTGACTGAG-3′ | (SEQ ID No. 13) |
| 5′-CGGCCCCCGAAGTCCCTGGGACG-3′ | (SEQ ID No. 14) |

A number of embodiments of the invention have been described. Nevertheless, it will be understood that various modifications may be made without departing from the spirit and scope of the invention. Accordingly, other embodiments are within the scope of the following claims.

What is claimed is:

1. A method for producing an encapsidated RNA virus, comprising the following steps:
(a) providing polypeptide coding sequences, wherein the polypeptides are capable of forming a capsid and packaging an RNA virus genomic sequence in a eukaryotic cell;
(b) providing a construct comprising RNA virus genomic sequences operably linked to a bacteriophage promoter and a bacteriophage transcription termination sequence, wherein the bacteriophage promoter and the bacteriophage transcription termination sequence are operably compatible;
(c) providing a coding sequence for a bacteriophage polymerase operably compatible with the bacteriophage promoter of step (b), wherein the coding sequence is operably linked to a poxvirus promoter; and,
(d) expressing the polypeptides of step (a), the RNA virus genomic sequences of step (b) and the coding sequence for a bacteriophage polymerase of step (c) together in a eukaryotic cell cytoplasm under conditions allowing for the expression of the sequences and assembly of a capsid comprising the RNA virus genomic sequences, thereby making an encapsidated RNA virus.

2. The method of claim 1, wherein the eukaryotic cell is an animal cell.

3. The method of claim 2, wherein the animal cell is a mammalian cell.

4. The method of claim 3, wherein the mammalian cell is a human cell.

5. The method of claim 1, wherein the genes encoding the capsid-forming polypeptides are cloned into a plasmid or a viral vector.

6. The method of claim 1, wherein the coding sequences of step (a) are operably linked to a promoter that is active in an animal cell cytoplasm.

7. The method of claim 1, wherein the RNA virus genomic sequence comprises an internal ribosomal entry site (IRES).

8. The method of claim 7, wherein the internal ribosomal entry site (IRES) is a hepatitis internal ribosomal entry site (IRES).

9. The method of claim 1, wherein the construct comprising RNA virus genomic sequences comprises a plasmid or a viral vector.

10. The method of claim 1, wherein the bacteriophage is selected from the group consisting of a T3 bacteriophage, a T7 bacteriophage and an SP6 bacteriophage.

11. The method of claim 10, wherein a T3 bacteriophage polymerase is expressed with a T3 bacteriophage promoter, a T7 bacteriophage polymerase is expressed with a T7 bacteriophage promoter and an SP6 bacteriophage polymerase is expressed with an SP6 bacteriophage promoter.

12. The method of claim 1, wherein the construct comprises a T3 bacteriophage transcription termination sequence and a T3 bacteriophage promoter, a T7 bacteriophage transcription termination sequence and a T7 bacteriophage promoter, or, an SP6 bacteriophage transcription termination sequence and a SP6 bacteriophage promoter.

13. The method of claim 3, wherein the promoter active in an animal cell cytoplasm is a promoter derived from a virus of the family Poxviridae.

14. The method of claim 13, wherein the virus of the family Poxviridae is a virus of the genus Orthopoxvirus.

15. The method of claim 14, wherein the virus of the genus Orthopoxvirus is a vaccinia virus.

16. The method of claim 15, wherein the vaccinia virus promoter is a late vaccinia virus promoter.

17. The method of claim 1, wherein the poxvirus is a virus of the Orthopoxvirus genus.

18. The method of claim 17, wherein the poxvirus of the Orthopoxvirus genus is a vaccinia virus.

19. The method of claim 1, wherein the poxvirus is a virus of a genus selected from the group consisting of a Parapoxvirus genus, Avipoxvirus genus, a Capripoxvirus genus, Yatapoxvirus genus, a Leporipoxvirus genus, a Suipoxvirus genus and a Molluscipoxvirus genus.

20. The method of claim 1, wherein the eukaryotic cell cytoplasm comprises a eukaryotic cell.

21. The method of claim 1, wherein the eukaryotic cell cytoplasm comprises an in vitro preparation.

22. The method of claim 1, wherein the RNA virus is a hepatitis virus comprising an RNA genome.

23. The method of claim 22, wherein the RNA virus is a hepatitis C virus.

24. The method of claim 22, wherein the RNA virus is an immature hepatitis B virus.

25. The method of claim 22, wherein the RNA virus is a hepatitis A virus.

26. The method of claim 1, wherein the RNA virus is a lentivirus.

27. The method of claim 1, wherein the RNA virus is a rhinovirus.

28. The method of claim 1, wherein the RNA virus is an influenza virus.

29. The method of claim 1, wherein the RNA virus is a human immunodeficiency virus (HIV).

30. The method of claim 29, wherein the human immunodeficiency virus (HIV) is HIV-1.

31. The method of claim 30, wherein the human immunodeficiency virus lacks a Rev-responsive element or an envelope sequence.

32. The method of claim 1, wherein the RNA virus is selected from the group consisting of an arenavirus, a LCMV, a parainfluenza virus, a reovirus, a rotavirus, an astrovirus, a filovirus, and a coronavirus.

33. The method of claim 1, wherein the coding sequence for a bacteriophage polymerase is cloned into a replication defective poxvirus.

34. The method of claim 1, wherein the replication defective, encapsidated RNA virus is infectious.

35. The method of claim 1, wherein the replication defective, encapsidated RNA virus is non-infectious.

36. The method of claim 1, wherein the method produces a preparation that is 99% free of replication competent poxvirus.

37. The method of claim 36, wherein the method produces a preparation that is 100% free of replication competent poxvirus.

38. The method of claim 1, wherein the replication defective poxvirus lacks the ability to make a polypeptide necessary for viral replication.

39. The method of claim 38, wherein the polypeptide necessary for viral replication is a viral capsid polypeptide.

40. The method of claim 1, wherein the replication defective poxvirus is defective because of a transcription activation or a transcriptional regulation defect.

41. The method of claim 1, wherein one, several or all of the polypeptide coding sequences of step (a) are incorporated into the RNA virus genomic sequence of step (b) and the construct further comprises an internal ribosomal entry site (IRES).

42. A system for producing an encapsidated RNA virus, comprising the following components:
(a) polypeptide coding sequences, wherein the polypeptides are capable of packaging an RNA virus genomic sequences and each coding sequence is cloned into a construct such that it is operably linked to a promoter;
(b) a construct comprising RNA virus genomic sequence operably linked to a bacteriophage promoter and a bacteriophage transcription termination sequence, wherein the RNA virus genomic sequence can be packaged into a capsid by the polypeptides of step (a);
(c) a coding sequence for a bacteriophage polymerase operably compatible with the bacteriophage promoter of step (b), wherein the coding sequence is operably linked to a poxvirus promoter; and,
wherein expressing the polypeptides of step (a), the RNA virus genomic sequence of step (b) and the coding sequence for a bacteriophage polymerase of step (c) together in a eukaryotic cell cytoplasm under conditions allowing for the expression of the coding sequences and assembly of a capsid comprising the RNA viral genomic sequence produces an encapsidated RNA virus.

43. The system of claim 42, wherein the eukaryotic cell is an animal cell.

44. The system of claim 43, wherein the animal cell is a mammalian cell.

45. The system of claim 44, wherein the mammalian cell is a human cell.

46. The system of claim 42, wherein the genes encoding the capsid-forming polypeptides are cloned into a plasmid or a viral vector.

47. The system of claim 42, wherein one, several or all of the polypeptide coding sequences of step (a) are incorporated into the RNA virus genomic sequence of step (b) and the construct further comprises an internal ribosomal entry site (IRES).

48. The system of claim 42, wherein the coding sequence for a bacteriophage polymerase is cloned into a replication defective poxvirus.

49. The system of claim 42, wherein the replication defective, encapsidated RNA virus is infectious.

50. The system of claim 42, wherein the replication defective, encapsidated RNA virus is non-infectious.

51. The system of claim 42, wherein the method produces a preparation that is 99% free of replication competent poxvirus.

52. The system of claim 51, wherein the method produces a preparation that is 100% free of replication competent poxvirus.

53. The system of claim 42, wherein the bacteriophage promoter is cloned into a replication defective poxvirus.

54. A recombinant viral genomic sequence comprising an RNA genomic sequence and a 2′,3′ cyclic phosphate at its 3′ end.

55. A recombinant viral particle comprising an RNA genomic sequence and a 2′,3′ cyclic phosphate at its 3′ end.

56. A recombinant viral genomic sequence comprising an RNA genomic sequence and a transcriptional terminator sequence for a bacteriophage RNA polymerase followed by a poly A sequence at its 3′ end.

57. A recombinant viral particle comprising an RNA genomic sequence and a transcriptional terminator sequence for a bacteriophage RNA polymerase followed by a poly A sequence at its 3′ end.

58. A recombinant lentivirus genomic sequence lacking a Rev-response element (RRE) or an envelope sequence and comprising a terminator sequence for a bacteriophage RNA polymerase.

59. A recombinant lentivirus particle comprising an RNA genomic sequence lacking a Rev-response element (RRE) or an envelope sequence and comprising a terminator sequence for a bacteriophage RNA polymerase.
